(19) United States
(12) Patent Application Publication
(10) Pub. No.: US 2009/0270799 A1
(43) Pub. Date: Oct. 29, 2009

(54) FLUID INJECTION DEVICE

(75) Inventors: Takeshi SETO, Chofu-shi (JP); Kazuo KAWASUMI, Chino-shi (JP)

Correspondence Address: OLIFF & BERRIDGE, PLC, P.O. BOX 320850, ALEXANDRIA, VA, 22320-4850, US

(73) Assignee: SEIKO EPSON CORPORATION, Tokyo (JP)

(21) Appl. No.: 12/422,564

(22) Filed: Apr. 13, 2009

(30) Foreign Application Priority Data
Apr. 24, 2008 (JP) 2008-113473

(51) Int. Cl. A61M 5/00

(52) U.S. Cl. 604/93.01

(57) ABSTRACT

A fluid injection device includes: a fluid injection unit having; a fluid chamber body in which a fixed face, a diaphragm facing the fixed face, a fluid chamber having a space surrounded by a side wall continuing the fixed face and the diaphragm, and an inlet channel tube and an outlet channel tube communicating with the fluid chamber are integrally molded; an actuator in close contact with the diaphragm; and a lid body holding the fluid chamber body, wherein inner surfaces of respective connecting parts of the fixed face, the side wall, and the diaphragm are rounded in molding, and a volume of the fluid chamber is reduced by the diaphragm, and a pulsating fluid is injected from a nozzle opening provided in an end direction of the outlet channel tube.

Japanese Patent Application No. 2008-113473 filed on Apr. 24, 2008, is hereby incorporated by reference in its entirety.

### BACKGROUND

1. Technical Field

The present invention relates to a fluid injection device of injecting a pulsating fluid by rapidly reducing a volume of a fluid chamber with a diaphragm.

2. Related Art

An operation using a fluid injected at a high speed is clinically applied to, especially, hepatectomy with difficulties in bleeding from microvessels or the like because an incision of organ parenchyma can be made while the vasculature such as blood vessels is maintained, further, the incidental damage on living tissues other than the incision is minor and the burden on patients is less, and the blood does not hinder the vision of the operative field due to little bleeding and quick operation can be performed.

As a surgical device using a fluid injected at a high speed, there is a fluid injection device of injecting a pulsating fluid from an opening provided in the end direction of an outlet channel tube by rapidly reducing a volume of a fluid chamber with a diaphragm (for example, see JP-A-2005-152127) . The fluid chamber (pump chamber) of the fluid injection device is formed by a space surrounded by the diaphragm, a pump chamber body, the outlet channel fixed part, and an inlet channel body.

In a structure of reducing the volume of the fluid chamber with the diaphragm, it is conceivable that, when air bubbles enter the fluid chamber from the outside or a gas within the fluid is emerged as air bubbles within the fluid chamber, if the volume of the fluid chamber is reduced, the pressure of the fluid chamber does not sufficiently rise due to the influence of the air bubbles and the pulsation of the fluid becomes weak.

According to the structure in JP-A-2005-152127, the fluid chamber is formed by the space formed by the plural members of the diaphragm, the pump chamber body, the outlet channel fixed part, and an inlet channel body. Thus, micro-gaps and hornlike corners are produced in the coupling parts of these members. In the case where the fluid is a liquid, it is expected that the gas entering the micro-gaps and corners or produced there is likely to stay.

### SUMMARY

An advantage of some aspects of the invention is to solve the problems described above and the invention can be realized in the following embodiments or application examples.

### APPLICATION EXAMPLE 1

A fluid injection device according to the application example including: a fluid injection unit having; a fluid chamber body in which a fixed face, a diaphragm facing the fixed face, a fluid chamber having a space surrounded by a side wall continuing the fixed face and the diaphragm, and an inlet channel tube and an outlet channel tube communicating with the fluid chamber are integrally molded; an actuator in close contact with the diaphragm; and a lid body holding the fluid chamber body, wherein inner surfaces of respective connecting parts of the fixed face, the side wall, and the diaphragm are rounded in molding, and a volume of the fluid chamber is reduced by the diaphragm, and a pulsating fluid is injected from a nozzle opening provided in an end direction of the outlet channel tube.

According to the application example, since the fluid chamber body in which a fluid (liquid) flows is formed by integrally molding the fluid chamber, the inlet channel tube, and the outlet channel tube and the interior of the fluid chamber is smoothly rounded, no micro-gaps or hornlike corners are formed due to coupling of plural members as in the related art. Therefore, air bubbles hardly stay in these micro-gaps and hornlike corners, and, even when gases exist in the liquid, they can be discharged with the liquid from the outlet channel tube. Thereby, the pressure of the fluid chamber is raised by reducing the volume according to the setting without being affected by the air bubbles within the fluid chamber, and stable high-speed injection of micro-droplets can be performed.

Further, since no micro-gaps or hornlike corners exist, the channel resistance in flowing within the fluid chamber can be reduced and the pressure loss due to the channel resistance can be reduced.

### APPLICATION EXAMPLE 2

In the fluid injection device according to the application example, it is preferable that the outlet channel tube is protruded on the fixed face, and the fluid chamber has a rotator shape and the outlet channel tube is provided near a rotational axis of the rotator shape, and a swirling flow generating part that generates a swirling flow of the fluid around the rotational axis of the rotator shape is provided on the inner surface of the fixed face or the diaphragm.

Here, as the swirling flow generating part, for example, grooves and projections formed on the fixed face or the inner surface of the diaphragm can be adopted.

In this manner, a swirling flow is generated by the swirling flow generating part in the fluid within the fluid chamber, and thereby, the fluid is pushed toward the outer circumference (toward the side wall) of the fluid chamber by the centrifugal force, the air bubbles having small specific gravity contained in the fluid are concentrated near the center of the swirling flow, i.e., near the rotational axis of the rotator shape, and the air bubbles can be eliminated from the outlet channel tube provided near the rotational axis of the rotator shape. Thus, the reduction in pressure amplitude due to the existence of air bubbles within the fluid chamber can be prevented, the influence on the reduction in volume by the air bubbles within the fluid chamber can be eliminated, and stable high-speed injection of micro-droplets can be performed.

### APPLICATION EXAMPLE 3

In the fluid injection device according to the application example, it is preferable that the inlet channel tube is provided on the fixed face in parallel to the outlet channel tube.

According to the configuration, the fixed face has a large area relative to the side wall, and there is an advantage that the degree of freedom of the arrangement of the inlet channel tube and the outlet channel tube and their mutual dimensions (especially, their diameters) is great.

### APPLICATION EXAMPLE 4

In the fluid injection device according to the application example, it is preferable that the inlet channel tube is protruded on the side wall.

According to the configuration, since the outlet channel tube is formed on the fixed face and the inlet channel is formed on the side wall, the outlet channel tube and the inlet channel tube face in the different directions from each other. Although details will be described later, a connecting tube that supplies the fluid is connected to the inlet channel tube. Accordingly, there is an advantage that the connecting tube is easily be connected.

### APPLICATION EXAMPLE 5

In the fluid injection device according to the application example, it is preferable that the outlet channel tube is provided on the fixed face, and the fluid chamber has a rotator shape and the outlet channel tube is provided near a rotational axis of the rotator shape, and the inlet channel tube is communicated along an inner circumferential surface of the side wall.

According to the configuration, the fluid supplied from the inlet channel tube swirls along the side wall inner surface of the fluid chamber. Therefore, the air bubbles contained in the fluid are concentrated near the rotational axis of the rotator shape as is the case of the above described structure of providing the swirling flow generating part, and the air bubbles can be eliminated from the outlet channel tube provided near the rotational axis of the rotator shape.

### APPLICATION EXAMPLE 6

In the fluid injection device according to the application example, it is preferable that the lid body includes an upper case holding the fixed face and a lower case holding a circumferential edge of the diaphragm, and projecting portions that respectively cover outer circumferences of side surfaces of the inlet channel tube and the outlet channel tube are provided on the upper case or the lower case.

In the fluid chamber body according to the application example, the fluid chamber including the fixed face, the diaphragm, and the side wall continuing the fixed face and the diaphragm, the inlet channel tube, and the outlet channel tube are integrally molded. Therefore, the respective partition walls forming the fluid chamber body have nearly the same thickness as that of the diaphragm, and the inlet channel tube and the outlet channel tube protruded from the fluid chamber do not have enough structural strength in use. Accordingly, the projecting portions covering the outer circumferences on the side surfaces of the inlet channel tube and the outlet channel tube are provided in the lid body, and thereby, the inlet channel tube and the outlet channel tube can be reinforced.

### APPLICATION EXAMPLE 7

In the fluid injection device according to the application example, it is preferable that the upper case is formed of a metal layer over the fixed face and the side wall, the metal layer being thicker than the diaphragm.

As described above, the fixed face, the side wall, and the diaphragm have nearly the same thickness. In this case, also the fixed face and the side wall are partition walls of thin plates, and accordingly, when the diaphragm is driven, also the fixed face and the side wall may deform and hinder the pressure rise within the fluid chamber. On this account, by forming the metal layer on the fixed face and the outer circumferential surface of the side wall, the structural strength of the fixed face and the side wall can be increased, and the deformation of the fixed face and the side wall can be suppressed and pressure loss can be prevented.

### APPLICATION EXAMPLE 8

In the fluid injection device according to the application example, it is desirable that the metal layer is formed over the fixed face, the side wall, and the circumferential edge of the diaphragm.

According to the configuration, since the deformation range of the diaphragm due to the actuator is controlled by the metal layer, unwanted deformation of the diaphragm when the pressure within the fluid chamber is high can be suppressed and the pressure fall can be prevented.

### APPLICATION EXAMPLE 9

In the fluid injection device according to the application example, it is preferable that the nozzle opening is formed on an end of the outlet channel tube.

According to the configuration, there is no need to form a nozzle opening by attaching a separate nozzle to the outlet channel tube as in the above described related art, and the structure can be simplified. Further, the fluid resistance in the channel from the fluid chamber to the nozzle opening can be reduced.

### APPLICATION EXAMPLE 10

In the fluid injection device according to the application example, it is desirable that a reinforcing tube that covers an outer circumferential side surface of the outlet channel tube is further provided.

Depending on the operative site, the distance from the fluid chamber to the nozzle opening may exceed 100 mm. In this regard, when the outlet channel tube is integrally formed with the fluid chamber, the outlet channel tube is an elongated thin tube and its structural strength may be insufficient. Accordingly, the outlet channel tube can be reinforced by providing the reinforcing tube.

### APPLICATION EXAMPLE 11

In the fluid injection device according to the application example, it is preferable that the inlet channel tube and the outlet channel tube are provided on the side wall in positions opposed with respect to the fluid chamber.

The fluid chamber (fluid chamber body) of the above described application example is a flat container member with the fixed face as an upper surface and the diaphragm as a bottom surface. Therefore, the thin fluid chamber body with no projecting portions in the thickness direction can be realized by providing the inlet channel tube and the outlet channel tube in positions opposed to each other on the side wall.

### APPLICATION EXAMPLE 12

In the fluid injection device according to the application example, it is preferable that the lid body has a nearly circular section shape in a perpendicular direction to a fluid flow direction.

In the above described fluid chamber body, the inlet channel tube, the fluid chamber, and the outlet channel tube can be arranged nearly in a line. Therefore, the fluid injection unit having a cylindrical shape can be formed by providing the section shape of the lid body in a nearly circular shape, and the fluid injection unit can be provided on an end of a thin tube like a catheter.

### APPLICATION EXAMPLE 13

In the fluid injection device according to the application example, it is preferable that the actuator is a thin-film piezoelectric element.

According to the configuration, the fluid injection unit can be further thinner to be inserted into vascular channels such as blood vessels.

### BRIEF DESCRIPTION OF THE DRAWINGS

The invention will be described with reference to the accompanying drawings, wherein like numbers reference like elements.

FIG. 1 is an explanatory diagram showing an example of a schematic configuration of a fluid injection device according to embodiment 1.

FIG. 2 is a sectional view showing a schematic structure of a fluid injection unit according to embodiment 1.

FIG. 3 is a sectional view showing A-A section in FIG. 2.

FIGS. 4A and 4B are sectional views showing an example of a method of manufacturing a fluid chamber body according to embodiment 1, and FIG. 4A shows a method of molding a matrix of the fluid chamber body and FIG. 4B shows a method of molding the fluid chamber body.

FIG. 5 is a sectional view showing a schematic structure of a fluid injection unit according to embodiment 2.

FIG. 6 is a sectional view showing A-A section in FIG. 5.

FIG. 7 is a plan view showing a schematic structure of a fluid injection unit according to embodiment 3.

FIG. 8 is a sectional view showing a schematic structure of a fluid injection unit according to embodiment 4.

FIG. 9 is a sectional view showing a relationship between the fluid chamber body and an upper case and a manufacturing method according to embodiment 4.

FIG. 10 is a sectional view showing a schematic structure of a fluid injection unit according to embodiment 5.

FIGS. 11A to 11D show a schematic structure of a fluid injection unit according to embodiment 6, and FIG. 11A is a sectional view along a liquid flow direction, FIG. 11B is a front view seen from an end T direction in FIG. 11A, FIG. 11C is a sectional view showing A-A section in FIG. 11A, and FIG. 11D is a sectional view showing B-B section in FIG. 11A.

### DESCRIPTION OF EXEMPLARY EMBODIMENTS

Hereinafter, embodiments of the invention will be described with reference to the drawings.

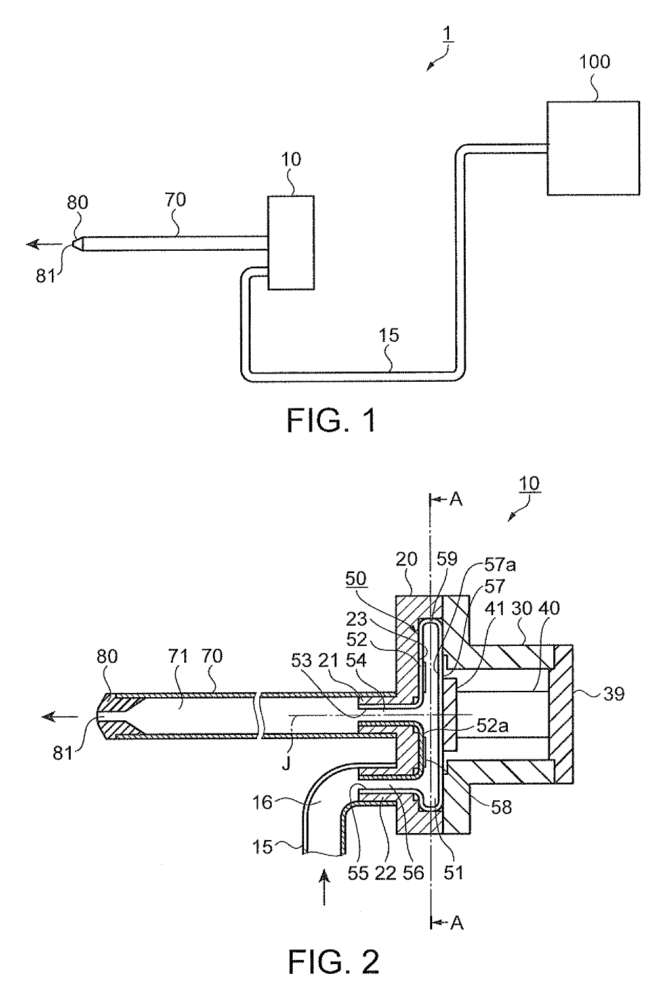

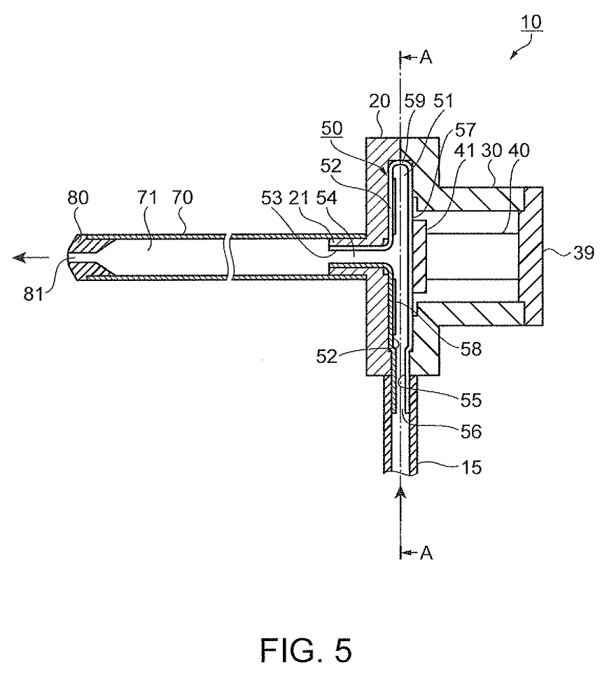

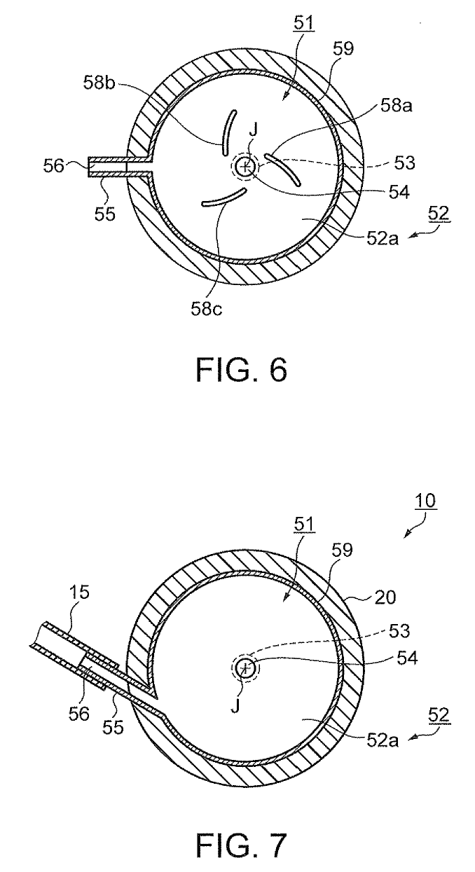

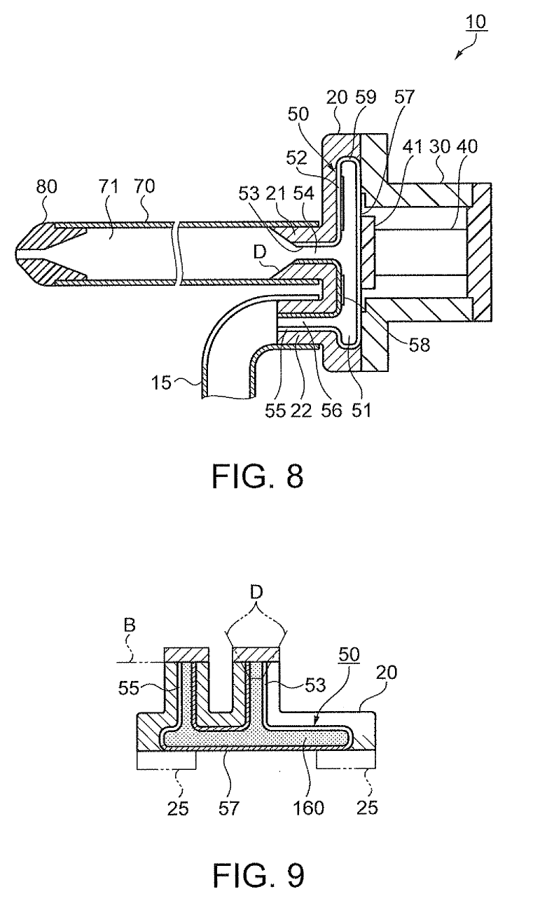

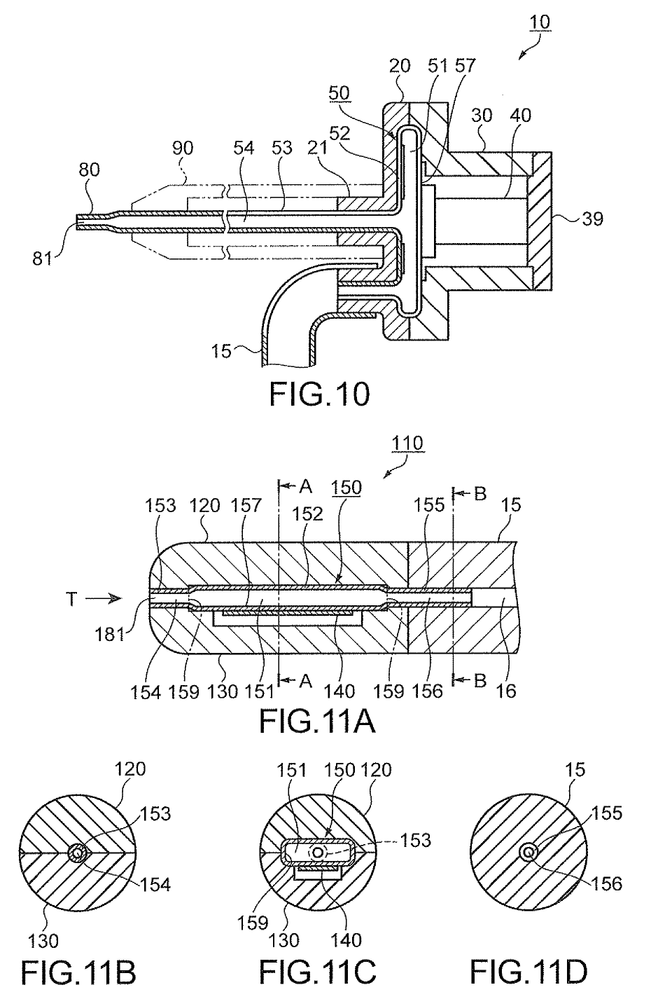

FIGS. 1 to 4B show a fluid injection device according to embodiment 1, FIGS. 5 and 6 show a fluid injection device according to embodiment 2, FIG. 7 shows a fluid injection device according to embodiment 3, FIGS. 8 and 9 show a fluid injection device according to embodiment 4, FIG. 10 shows a fluid injection device according to embodiment 5, and FIGS. 11R to 11D show a fluid injection device according to embodiment 6.

The drawings referred to in the following description are schematic diagrams in which horizontal and vertical scale of members and parts differs from the actual scale for convenience of illustration.

Further, a fluid injection device of the invention is adaptable to various uses for drawing using ink or the like, cleaning of minute objects and structures, surgical scalpels, etc. In the embodiments described as below, a fluid injection device preferable for a fluid injection device suitable for incision or excision of living tissues or being provided on a tip of a catheter inserted into a blood vessel for the purpose of removing a blood clot or the like will be described as an example. Accordingly, the fluid used in the following embodiments is water or normal saline solution, and that is referred to as a fluid and described as below.

### Embodiment 1

First, an overall configuration of the fluid injection device will be described.

FIG. 1 is an explanatory diagram showing an example of a schematic configuration of the fluid injection device according to embodiment 1. In FIG. 1, the fluid injection device 1 has a control unit 100 that includes an infusion bag as a fluid supply part containing a liquid for supply of the liquid, a fluid injection unit 10 that changes the liquid into pulsation, and a connecting tube 15 that communicates the control unit 100 with the fluid injection unit 10 as a basic configuration. A connecting channel tube 70 in a thin pipe shape is connected to the fluid injection unit 10 and a nozzle 80 having a nozzle opening 81 at which the channel is reduced is inserted at the end of the connecting channel tube 70. The fluid injection unit 10 changes the liquid into pulsation and injects the pulsating liquid via the connecting channel tube 70 and the nozzle 80 as droplets at a high speed from the nozzle opening 81.

The control unit 100 includes a drive waveform generation circuit part and a drive control circuit part (not shown) and an adjusting device that adjusts a drive waveform according to a condition such as hardness of an operative site.

Subsequently, a flow of the liquid in the fluid injection device 1 will be briefly described. The liquid contained in the infusion bag is supplied to the fluid injection unit 10 at fixed pressure via the connecting tube 15. The control unit 100 includes a pressure generating part (not shown) of a pump or the like that supplies the liquid at the fixed pressure. The fluid injection unit 10 includes a fluid chamber 51 (see FIG. 2) and volume changing means of the fluid chamber 51, and generates pulsation with the volume changing means and injects the pulsating liquid via the connecting channel tube 70 and the nozzle 80 at a high speed from the nozzle opening 81.

The pressure generating part is not limited to a pump, but the infusion bag may be held at a higher position than that of the fluid injection unit 10 with a stand or the like. Accordingly, the pump is not necessary, and there are advantages that the configuration can be simplified and sterilization and the like becomes easier.

The liquid supply pressure in the pressure generating part is set to generally equal to or less than three atmospheres, desirably equal to or less than 0.3 atmospheres (0.03 MPa). Further, when the infusion bag is used, the pressure is provided as a height difference between the fluid injection unit 10 and the liquid level of the infusion bag. When the infusion bag is used, the height difference is desirably set so that the pressure may be about 0.1 to 0.15 atmospheres (0.01 to 0.15 MPa).

When an operation is performed using the fluid injection device 1, the site grasped by an operator is fluid injection unit 10. Accordingly, it is preferable that the connecting tube 15 to the fluid injection unit 10 is as flexible as possible. For that, it is preferable that the connecting tube 15 is a flexible thin tube and the pressure is set to low pressure in a range in which the liquid can be sent to the fluid injection unit 10.

Further, especially, when an instrumental malfunction may cause a serious accident as in a brain operation, injection of a high-pressure fluid due to breakage of the connecting tube 15 or the like should be avoided, and this requires low pressure.

Next, a structure of the fluid injection unit according to the embodiment will be described.

FIG. 2 is a sectional view showing a schematic structure of the fluid injection unit according to the embodiment. In FIG. 2, the fluid injection unit 10 has a fluid chamber body 50 including the fluid chamber 51 as pulsation generating means that generates pulsation of the liquid and a piezoelectric element 40 as an actuator, and an upper case 20 and a lower case 30 as a lid body that holds the fluid chamber body 50.

In the fluid chamber body 50, a fixed face 52, a diaphragm 57 facing the fixed face 52, the fluid chamber 51 of a space formed by being surrounded by a side wall 59 continuing the fixed face 52 and the diaphragm 57, and an inlet channel tube 55 and an outlet channel tube 53 communicating with the fluid chamber 51 are integrally molded using the same material. Accordingly, the fluid chamber body 50 may be considered to be in a flat container shape. The inlet channel tube 55 and the outlet channel tube 53 are provided perpendicularly to the fixed face 52 in parallel. Further, the inlet channel tube 55 has an inlet channel 56 and the outlet channel tube 53 has an outlet channel 54, and they communicate with the interior of the fluid chamber 51.

The fixed face 52, the side wall 59, the diaphragm 57, the inlet channel tube 55, and the outlet channel tube 53 are integrally formed and the inner surfaces of the respective connecting parts are smoothly rounded. As a result, there are no micro-gaps or hornlike corners due to coupling of plural members as in the related art.

The fluid chamber body 50 is fitted into a concave portion 23 recessed in the upper case 20, and the fixed face 52 and the side wall 59 are fixed to the upper case 20 in close contact using an adhesive or the like within the concave portion 23. Tubular projecting portions 21, 22 are provided in the upper case 20, and the outlet channel tube 53 and the inlet channel tube 55 are inserted into the projecting portions 21, 22, respectively. The outlet channel tube 53 and the inlet channel tube 55 have weak structural strength because they have nearly the same thickness as that of the diaphragm 57, and are reinforced by the projecting portions 21, 22.

The projecting portion 21 is inserted into the connecting channel tube 70 and a connecting channel 71 opened in the connecting channel tube 70 communicates with the outlet channel 54. Further, the nozzle 80 is fitted in the end of the connecting channel tube 70 and the nozzle opening 81 having a reduced diameter compared to that of the outlet channel 54 is provided at the tip of the nozzle 80. Furthermore, the connecting tube 15 is fitted in the projecting portion 22 and a liquid flowing part 16 is communicated with the inlet channel 56.

On the other hand, the lower case 30 having a through hole for holding the piezoelectric element 40 is provided at the diaphragm 57 side of the fluid chamber body 50, and the upper case 20 and the lower case 30 are fastened in close contact using coupling means such as bonding or screwing on their opposed circumferential edge surfaces. In this regard, part of the circumferential edge surface of the lower case 30 reaches the circumferential edge surface of the diaphragm 57 and is fixed to the circumferential edge surface of the diaphragm 57 using fixing means such as bonding. The fixed range of the lower case 30 and the diaphragm 57 includes a margin left for allowing necessary displacement of the diaphragm 57.

The piezoelectric element 40 as an actuator is in close contact with the central part of the diaphragm 57 with an upper plate 41 in between, and the tail portion side of the piezoelectric element 40 is fixed to a lower plate 39. The lower plate 39 is fixed to the lower case 30. Accordingly, the lower plate 39, the piezoelectric element 40, the upper plate 41 and the diaphragm 57 are in close contact with each other. A multilayered piezoelectric element is taken as an example of the piezoelectric element 40 in the embodiment. Further, a swirling flow generating part 58 is formed on a fluid chamber inner surface 52a of the fixed face 52.

Subsequently, an example of the swirling flow generating part will be described with reference to the drawings.

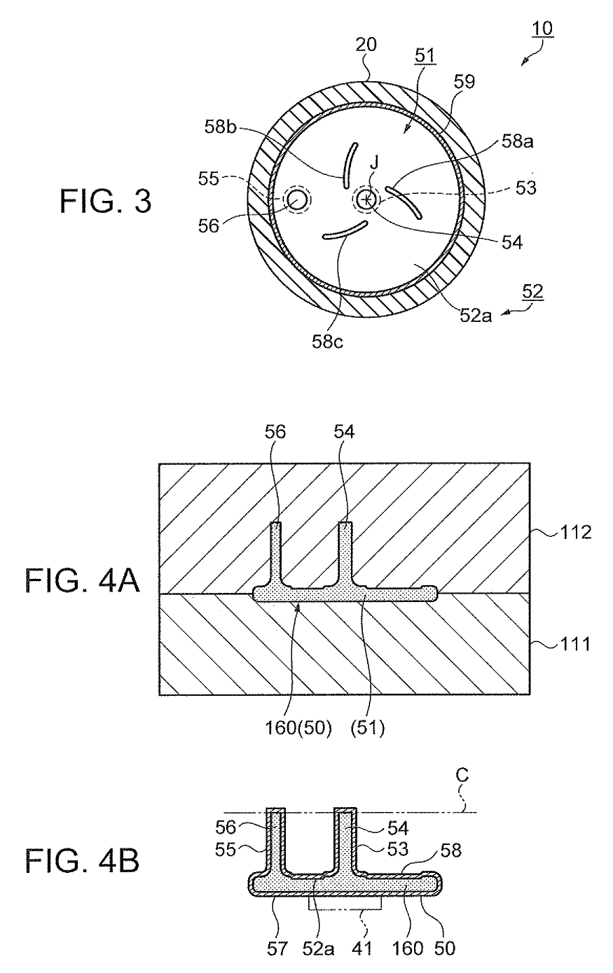

FIG. 3 is a sectional view showing A-A section in FIG. 2. In FIG. 3, the swirling flow generating part 58 includes projecting portions 58a, 58b, 58c curved toward the flat surface provided on the surface of the fluid chamber inner surface 52a. The projecting portions 58a to 53c are curved in the same direction and provided between the outlet channel 54 and the inlet channel 56. Further, as shown in FIG. 3, the fluid chamber 51 has a rotator shape and the outlet channel tube 53 is provided near the rotational axis J of the rotator shape.

The projecting portions 58a to 58c forming the swirling flow generating part 58 may be provided on the inner surface 57a of the diaphragm 57 (see FIG. 2). Further, they may be grooves curved relative to the projecting portions. Furthermore, the number of projecting portions may be appropriately larger or smaller than three.

Next, an example of a method of manufacturing the fluid chamber body 50 will be described with reference to the drawings.

FIGS. 4A and 4B are sectional views showing an example of a method of manufacturing the fluid chamber body 50 according to the embodiment, and FIG. 4A shows a method of molding a matrix of the fluid chamber body and FIG. 4B shows a method of molding the fluid chamber body. First, as shown in FIG. 4A, a fluid chamber body matrix 160 is molded using a mold. The fluid chamber body matrix 160 is molded using an upper mold 112 and a lower mold 111 according to the resin molding method (injection molding method). The outer shape of the fluid chamber body matrix 160 includes a part corresponding to the fluid chamber 51 and a part corresponding to the inlet channel 56 and the outlet channel 54. Accordingly, the outer shape of the fluid chamber body matrix 160 has an inner surface shape of the fluid chamber body 50.

In the fluid chamber body matrix 160, since the part corresponding to the inlet channel 56 and the outlet channel 54 has an elongated shape, drawing tapers are formed in the upper mold 112. Accordingly, the respective sectional areas of the inlet channel 56 and the outlet channel 54 at the fluid chamber 51 side are larger than the sectional areas at the ends side by the areas of the drawing tapers.

In the flowing direction of the liquid within the thin tube, when the sectional area at the liquid inflow side is smaller than that at the liquid outflow side, the liquid is made easier to flow because of the diffusion effect and the liquid is made harder to flow because of the nozzle effect in the liquid flow in the opposite direction. Accordingly, the flow to the fluid chamber 51 can be promoted because of the diffusion effect in the inlet channel 56, and the flow from the fluid chamber 51 (i.e., backflow) can be suppressed because of the nozzle effect.

On the other hand, in the outlet channel 54, when the volume of the fluid chamber 51 is returned from the reduced state and the internal pressure is reduced, the liquid existing within the outlet channel 54 becomes easily to be drawn back into the fluid chamber 51 because of the diffusion effect and unwanted droplet discharge can be suppressed between drive signal outputs.

Subsequently, as shown in FIG. 4B, electroless Ni plating is performed on the entire surface of the fluid chamber body matrix 160. The thickness of the plated layer refers to the thickness of the diaphragm 57. In this manner, an original form of the fluid chamber body 50 made of Ni is formed. In this regard, since the plated layer is formed on the entire surface of the fluid chamber body matrix 160, the layer is also formed on the ends of the parts corresponding to the inlet channel 56 and the outlet channel 54. By cutting along the cutting surface C, the resin parts are exposed on the ends.

As the method of forming the fluid chamber body 50, not only by electroless Ni plating, but also a sputtering method on the surface of the fluid chamber body matrix 160 may be used. In the sputtering method, a wide variety of materials such as titanium can be adopted other than Ni as a metal layer. Further, other materials that satisfy the function as the diaphragm may be used in the case of using the plating method.

Then, the fluid chamber body matrix 160 is dissolved and removed using a solvent, the interior is cleaned, and only the fluid chamber body 50 (see FIG. 2) is left. In this manner, the fluid chamber body 50 is molded. It is desirable that adjustment is made by performing heat treatment (hardening treatment) so that, especially, the appropriate hardness and elasticity as the diaphragm may be provided after the fluid chamber body 50 is molded.

Subsequently, an operation of the fluid injection unit 10 will be described with reference to FIG. 2.

The liquid is supplied to the inlet channel 56 constantly at fixed liquid pressure by the pressure generating part. As a result, when the piezoelectric element 40 does not move, the liquid flows into the fluid chamber 51 according to the difference between the discharge force of the pressure generating part and the fluid resistance value of the entire inlet channel side.

Here, if the drive signal is input to the piezoelectric element 40 and the piezoelectric element 40 rapidly expands, the pressure within the fluid chamber 51 promptly rises and reaches several tens of atmospheres when the inertance L1 at the inlet channel side and the inertance L2 at the outlet channel side have sufficient magnitudes.

Since the pressure is far greater than the pressure applied to the inlet channel 56 by the pressure generating part, the liquid flow from the inlet channel 56 into the fluid chamber 51 is reduced due to the pressure and the outflow from the outlet channel 54 is increased.

However, the inertance L1 at the inlet channel side is larger than the inertance L2 at the outlet channel side, the reduced amount of flow of the liquid from the inlet channel 56 into the fluid chamber 51 is smaller than the increased amount of the liquid discharged from the outlet channel 54, and therefore, the pulsating liquid discharge, i.e., the pulsating flow is generated in the connecting channel 71. The pressure variation at discharge propagates within the connecting channel tube 70, and the liquid is injected from the nozzle opening 81 at the tip.

Here, since the diameter of the nozzle opening 81 is made smaller than the diameter of the outlet channel 54, the liquid is injected as pulsing droplets at higher pressure at a higher speed.

On the other hand, the pressure within the fluid chamber 51 turns into a low pressure or vacuum state immediately after rise because of the interaction of the reduction in the inflow amount of the liquid from the inlet channel 56 and the increase in the outflow amount of the liquid from the outlet channel 54. As a result, the liquid in the inlet channel 56 returns to the flow into the fluid chamber 51 at the same speed as that before the movement of the piezoelectric element 40 after a certain period of time due to both the pressure of the pressure generating part and the pressure within the fluid chamber 51.

After the flow of the liquid within the inlet channel 56 returns, if there is expansion of the piezoelectric element 40, the pulsating droplets can be continuously injected from the nozzle opening 81.

Subsequently, an elimination operation of air bubbles within the fluid chamber 51 will be described with reference to FIG. 3. In the embodiment, it is considered that no coupling parts of the component members exist on the inner surface of the fluid chamber 51 and smoothly rounded and minute air bubbles hardly stay in the micro-gaps and the hornlike corners of the coupling part of the members, however, it is conceivable that gasses may be contained in the liquid.

In the above described operation of the fluid injection unit 10, the fluid chamber 51 has a rotator shape and includes the projecting portions 58a to 58c as the swirling flow generating part 58, and accordingly, in the liquid flowing from the inlet channel 56 at fixed pressure, a clockwise swirling flow around the rotational axis J is generated by the projecting portions 58a to 58c. The liquid is swirled along the side wall due to the centrifugal force generated by the swirling flow. Concurrently, the air bubbles generated at inflow or from fluid gather near the rotational axis J of the rotator shape. Then, the air bubbles are immediately discharged from the outlet channel 54 to the outside because the outlet channel 54 is opened near the rotational axis J.

Therefore, according to the embodiment, in the fluid chamber body 50, the fluid chamber 51, the inlet channel tube 55, and the outlet channel tube 53 are integrally molded and the interior of the fluid chamber 51 is smoothly rounded, and thus, no micro-gaps or hornlike corners due to coupling of plural members are formed as in the related art. Accordingly, the air bubbles hardly stay in these micro-gaps and the hornlike corners, and, even when air bubbles exist in the liquid, they can be discharged with the liquid from the outlet channel 54. Thereby, the volume is reduced according to the setting without being affected by the air bubbles within the fluid chamber 51, and stable high-speed injection of micro-droplets can be performed.

Further, since no micro-gaps or hornlike corners exist, the fluid resistance of the liquid flowing within the fluid chamber 51 can be reduced and the pressure loss due to the channel resistance can be reduced.

Furthermore, a swirling flow is generated within the fluid chamber 51 by the swirling flow generating part 58 (projecting portions 58a to 58c) within the fluid chamber 51, and thereby, the liquid is pushed toward the outer circumference (toward the side wall 59) of the fluid chamber 51 by the centrifugal force, the air bubbles gather near the center of the swirling flow, i.e., near the rotational axis J of the rotator shape, and the air bubbles can be eliminated with the liquid from the outlet channel 54 provided near the rotational axis J. Thus, the reduction in pressure amplitude due to the existence of air bubbles within the fluid chamber 51 can be prevented and stable high-speed injection of micro-droplets can be performed.

Moreover, the inlet channel tube 55 and the outlet channel tube 53 are provided on the fixed surface 52 in parallel. According to the configuration, the fixed surface 52 has a large area relative to the side wall 59, and there is an advantage that the degree of freedom of the layout of the inlet channel tube 55 and the outlet channel tube 53 and their mutual dimensions (especially, their diameters) is great.

In addition, the respective partition walls forming the inlet channel tube 55 and the outlet channel tube 53 have nearly the same thickness as that of the diaphragm 57, and the inlet channel tube 55 and the outlet channel tube 53 protruded from the fluid chamber 51 do not have enough structural strength in use. Accordingly, the projecting portions 21, 22 covering the outer circumferences on the side surfaces of the inlet channel tube 55 and the outlet channel tube 53 are provided in the upper case 20, and thereby, the inlet channel tube 55 and the outlet channel tube 53 can be reinforced.

### Embodiment 2

Subsequently, a fluid injection device according to embodiment 2 will be described with reference to the drawings. Embodiment 2 is characterized by providing the inlet channel tube on the side wall of the fluid chamber. Accordingly, the description is centered on the differences from the above described embodiment 1 (see FIG. 1) and the same signs are assigned to the same functional parts.

FIG. 5 is a sectional view showing a schematic structure of a fluid injection unit according to embodiment 2, and FIG. 6 is a sectional view showing A-A section in FIG. 5. In FIGS. 5 and 6, the fluid chamber body 50 includes the outlet channel tube 53 protruded perpendicularly to the fixed face 52 and the inlet channel tube 55 protruded on the side will 59 of the fluid chamber body 50 (fluid chamber 51). The outlet channel tube 53 has the outlet channel 54 and the inlet channel tube 55 has the inlet channel 56, and the respective tubes communicate with the fluid chamber 51.

The inlet channel tube 55 is protruded on the lid body including the upper case 20 and the lower case 30, and the connecting tube 15 is fitted in the end. The inlet channel 56 has a circular shape with a sectional area perpendicular to the liquid flow direction made smaller than the sectional area of the fluid chamber 51. The fluid chamber body 50 is held by the upper case 20 and the lower case 30.

As shown in FIG. 6, the fluid chamber 51 has a rotator shape and the inlet channel tube 55 communicated toward the rotational axis J of the rotator shape, and thereby, the liquid is allowed to flow from the inlet channel 56 toward the rotational axis J. Here, the swirling flow generating part 58 including projecting portions 58a, 58b, 58c curved toward the flat surface is formed on the fluid chamber inner surface 52a of the fixed face 52 of the fluid chamber 51 (also see FIG. 5). Accordingly, the liquid flowing from the inlet channel 56 at fixed pressure is swirled along the side wall due to the centrifugal force in the clockwise direction around the rotational axis J of the rotator shape by the swirling flow generating part 58. Concurrently, the air bubbles gather near the rotational axis J. The air bubbles are immediately discharged from the outlet channel 54 to the outside because the outlet channel 54 is communicated near the rotational axis J.

Therefore, according to the configuration, since the outlet channel tube 53 is formed on the fixed face 52 of the fluid chamber 51 and the inlet channel tube 55 is formed on the side wall 59, the outlet channel tube 53 and the inlet channel tube 55 face in the different directions from each other (directions nearly perpendicular to each other). The connecting tube 15 that supplies the liquid is connected to the inlet channel tube 55. Accordingly, the connecting tube 15 is easily be connected, and further, it is not necessary to bend the connecting tube 15 in another direction near the connecting part as in embodiment 1 (see FIG. 2).

It is more preferable that the projecting portions covering around the inlet channel tube 55 described in embodiment 1 are provided as tubular reinforcing portions in the respective upper case 20 and lower case 30 and the connecting tube 15 is fitted in the reinforcing portions.

Further, the fluid chamber body 50 in the embodiment can be molded according to the same manufacturing method as that in the above described embodiment 1 (see FIGS. 4A and 4B).

### Embodiment 3

Subsequently, a fluid injection device according to embodiment 3 will be described with reference to the drawings. Embodiment 3 is characterized by communicating the inlet channel with the fluid chamber along the inner circumferential surface of the side wall. Accordingly, the description is centered on the differences from the above described embodiment 2 (see FIGS. 5, 6).

FIG. 7 is a plan view showing a schematic structure of a fluid injection unit according to embodiment 3 corresponding to the A-A section in FIG. 5. In FIG. 7, the inlet channel tube 55 is protruded so that the inlet channel 56 may communicate along the inner circumferential surface of the side wall 59 of the fluid chamber 51.

The fluid chamber 51 has a rotator shape, and the outlet channel tube 53 is protruded perpendicularly to the fixed face 52 near the rotational axis J of the rotator shape.

The liquid flowing from the inlet channel 56 at fixed pressure flows along the inner circumferential surface of the side wall 59 within the fluid chamber 51, and swirls in the counter-clockwise direction in the configuration shown in FIG. 7. Concurrently, the air bubbles contained in the liquid gather near the rotational axis J, and the air bubbles are immediately discharged from the outlet channel 54 to the outside because the outlet channel 54 is opened near the rotational axis J.

It is more preferable that the projecting portions covering around the inlet channel tube 55 described in embodiment 1 are provided as tubular reinforcing portions in the respective upper case 20 and lower case 30 and the connecting tube 15 is fitted in the reinforcing portions.

Further, the fluid chamber body 50 in the embodiment can be molded according to the same manufacturing method as that in the above described embodiment 1 (see FIGS. 4A and 4B).

According to the configuration, the liquid flowing from the inlet channel 56 swirls along the inner circumference of the side wall 59 of the fluid chamber 51. Therefore, even with the structure simpler than the structure in which the above described swirling flow generating part 58 including projecting portions 58a, 58b, 58c is provided, the air bubbles contained in the liquid are concentrated near the rotational axis J, and can be eliminated from the outlet channel 54 provided near the rotational axis J.

### Embodiment 4

Subsequently, a fluid injection device according to embodiment 4 will be described with reference to the drawings. Embodiment 4 is characterized by having a different configuration between the fluid chamber body and the upper case from that of the above described embodiment 1 to embodiment 3. Accordingly, the description is centered on the differences from the embodiment 1 to embodiment 3 and the same signs are assigned to the same functional parts.

FIG. 8 is a sectional view showing a schematic structure of a fluid injection unit according to the embodiment, and FIG. 9 is a sectional view showing a relationship between the fluid chamber body and the upper case and a manufacturing method. FIGS. 8 and 9 show the case of adopting the basic structure of the above described embodiment 1 (see FIG. 2) as an example. In FIGS. 8 and 9, the upper case 20 is formed on the outer circumferential surface of the fixed face 52 and the side wall 59 of the fluid chamber body 50 by a thicker metal layer than the diaphragm 57. The metal layer is formed in close contact along the entire circumference from the side wall 59 to the fixed face 52, the side surface of the inlet channel tube 55 and the outlet channel tube 53 of the fluid chamber body 50 by electroless Ni plating or the like, and forms the upper case 20 as shown in FIG. 8.

More specifically, as shown in FIG. 9, in the process of the manufacturing method of the fluid chamber body 50 described in embodiment 1 (see FIGS. 4A and 4B) (under the condition that the fluid chamber body matrix 160 is loaded within the fluid chamber body 50), the metal plating layer is further formed. Accordingly, the predetermined surface of the fluid chamber body 50 is coated with the metal layer in a homogeneous thickness, and thereby, the upper case 20 is formed. Then, a part of the fluid chamber body matrix 160 is exposed by cutting the case along the cutting surface (shown by a chain double-dashed line B) of the inlet channel tube 55.

In the outlet channel tube 53, a part of the fluid chamber body matrix 160 is exposed by forming slopes D from the end by cutting. Then, the fluid chamber body matrix 160 is dissolved and removed using a solvent and the interior is cleaned, and the fluid chamber body 50 and the upper case 20 are integrally formed. In this manner, as shown in FIG. 8, when the fluid chamber body 50 is connected to the connecting channel tube 70, the outlet channel tube 53 and the side partition wall of the connecting channel tube 70 are gently continued by the slopes D.

The metal layer forming the upper case 20 may extend to the circumferential edge of the diaphragm 57. That is, a metal layer 25 is formed by extending from the side surface to the circumferential edge except the appropriate range as the diaphragm 57. The metal layer 25 may be formed with masking in a predetermined range of the diaphragm 57.

When the fluid chamber body 50 is formed as described above, the fluid chamber body 50 has nearly the same thickness as those of the fixed face 52, the side wall 59, and the diaphragm 57. In the configuration, also the fixed face 52 and the side wall 59 are partition walls of thin plates, and accordingly, when the diaphragm 57 is driven, also the fixed face 52 and the side wall 59 may deform, the fluid chamber may deform, and the pressure amplitude may be reduced. On this account, by forming the metal layer along the fixed face 52 and the outer circumferential surface of the side wall 59, the structural strength of the fixed face 52 and the side wall 59 can be increased, and the deformation of the fixed face 52 and the side wall 59 can be suppressed and cavitation can be prevented from occurring.

Further, since the metal layer 25 is formed over the outer circumferential surface of the fixed face 52 and the side wall 59 and the circumferential edge of the diaphragm 57, the deformation range of the diaphragm 57 due to the piezoelectric element 40 is controlled by the metal layer 25, and the amount of volume change of the fluid chamber 51 can be maintained constant.

Further, since the outlet channel tube 53 and the side partition wall of the connecting channel tube 70 are gently continued by the slopes D under the condition that the connecting channel tube 70 is connected to the fluid chamber body 50, pressure loss due to abrupt expansion can be suppressed between the nozzle 80 and the outlet channel tube 53.

As the method of forming the upper case 20, it can be formed by molding the outer shape of the fluid chamber body 50 as a matrix using a resin, forming the metal layer by means such as electroless Ni plating and sputtering on the surface of the matrix, and dissolving the matrix. According to the forming method, the opposed surfaces of the upper case 20 and the fluid chamber body 50 are in close contact. Therefore, by fastening the upper case 20 and the fluid chamber body 50 with an adhesive, the same advantage as that of the above described configuration in which the upper case 20 and the fluid chamber body 50 are integrally formed can be obtained.

### Embodiment 5

Subsequently, a fluid injection device according to embodiment 5 will be described with reference to the drawings. Embodiment 5 is characterized in that the nozzle 80 is integrally formed on the end of the outlet channel tube 53 while the connecting channel tube 70 having the nozzle 80 is connected to the end of the outlet channel tube 53 in the above described fluid injection unit 10 according to embodiment 1 to embodiment 4. Embodiment 1 (see FIG. 2) is illustrated as the basic configuration and the description is centered on the differences. The same signs are assigned to the same functional parts.

FIG. 10 is a sectional view showing a schematic structure of a fluid injection unit according to embodiment 5. In FIG. 10, the outlet channel tube 53 is further protruded and extended relative to the projecting portion 21 of the fluid chamber body 50 in the perpendicular direction from the fixed face 52 of the fluid chamber body 50.

On the end, the nozzle 80 having the nozzle opening 81 with a sectional area perpendicular to the liquid flow direction made smaller than that of the outlet channel 54 is formed.

Further, a reinforcing tube 90 covering the outer circumferential side surface of the outlet channel tube 53 is further provided. The end of the reinforcing tube 90 supports the part near the nozzle 80 and the base thereof is fitted in the projecting portion 21.

Therefore, in the configuration according to the embodiment, the outlet channel tube 53 is extended longer and the nozzle 80 (nozzle opening 81) is formed on the end, and thereby, it is not necessary to attach the connecting channel tube 70 and the nozzle 80 to the outlet channel tube 53 and form the nozzle opening 81 and the structure can be simplified. Further, the fluid resistance in the channel from the fluid chamber 51 to the nozzle opening 81 can be reduced.

Furthermore, when the fluid injection device is used as a surgical instrument, the distance from the fluid chamber 51 to the nozzle opening 81 may exceed 100 mm depending on the operative site. In this regard, the outlet channel tube 53 is an elongated thin tube and its structural strength may be insufficient. Accordingly, the outlet channel tube 53 can be reinforced by providing the reinforcing tube 90.

### Embodiment 6

Subsequently, a fluid injection device according to embodiment 6 will be described with reference to the drawings. Embodiment 6 is characterized by providing the inlet channel tube and the outlet channel tube in positions opposed relative to the fluid chamber on the side wall forming the fluid chamber.

FIGS. 11A to 11D show a schematic structure of a fluid injection unit according to embodiment 6. FIG. 11A is a sectional view along the liquid flow direction, FIG. 11B is a front view seen from the end T direction in FIG. 11A, FIG. 11C is a sectional view showing A-A section in FIG. 11A, and FIG. 11D is a sectional view showing B-B section in FIG. 11A. In FIGS. 11A to 11D, a fluid injection unit 110 includes a fluid chamber body 150 and a lid body having an upper case 120 and a lower case 130 holding the fluid chamber body 150.

In the fluid chamber body 150, a fixed face 152, a diaphragm 157 facing the fixed face 152, a fluid chamber 151 formed by being surrounded by a side wall 159 continuing the fixed face 52 and the diaphragm 157, and an inlet channel tube 155 and an outlet channel tube 153 communicating with the interior of the fluid chamber 151 are integrally molded using the same material. The inlet channel tube 155 and the outlet channel tube 153 are protruded in positions opposed relative to the fluid chamber 151 perpendicularly to the side wall 159 in the liquid flow direction. Accordingly, the fluid chamber 151 may be considered to be a flat container elongated in the liquid flow direction.

Further, the inlet channel tube 155 has an inlet channel 156 and the outlet channel tube 153 has an outlet channel 154, and they communicate with the fluid chamber 151. Both the inlet channel tube 155 and the outlet channel tube 153 are formed by thin tubes having circular sections. The end of the outlet channel tube 153 is a nozzle opening 181 and the sectional area is smaller than the sectional area of the outlet channel 154 perpendicular to the liquid flow direction.

The inner surface of the fluid chamber 151 and the inner surface of the connecting parts of the inlet channel tube 155 and the outlet channel tube 153 and the fluid chamber 151 are smoothly rounded.

A thin-film piezoelectric element 140 as an actuator is fixed in close contact with the outer surface of the diaphragm 157. The shape of the thin-film piezoelectric element 140 is a rectangular along the flat surface shape of the diaphragm 157.

The fluid chamber body 150 is held between the upper case 120 and the lower case 130 as the lid body. The fixed face 152 of the fluid chamber body 150 is fixed to the upper case 120 and the circumferential edge of the diaphragm 157 is fixed to the lower case 130. Here, the section shape of the lid body in the perpendicular direction to the liquid flow direction is nearly circular when the upper case 120, the fluid chamber body 150, and the lower case 130 are integrated.

The end of the inlet channel tube 155 is projected from the lid body and the connecting tube 15 is connected thereto. The connecting tube 15 has an outer diameter nearly equal to the diameter of the lid body and is inserted into the inlet channel tube 155 into contact with the end of the lid body.

According to the configuration, since the inlet channel tube 155 and the outlet channel tube 153 are provided in positions opposed relative to the fluid chamber 151 on the side wall 159, respectively, the thin fluid chamber body 150 with no projecting portions in the thickness direction can be realized.

Further, since the lid body including the upper case 120 and the lower case 130 and holding the fluid chamber body 150 has a nearly circular sectional shape in the perpendicular direction to the liquid flow direction, the fluid injection unit 110 having a cylindrical shape can be formed, and the fluid injection unit can be provided on an end of a thin tube like a catheter.

Furthermore, by adopting the thin-film piezoelectric element 140 as an actuator, the fluid injection unit 110 can be made further thinner to be inserted into vascular channels such as blood vessels. In this regard, the outer diameter of the connecting tube 15 and the lid body are made equal, and thereby, the unit can be prevented from damaging tissues when inserted into or withdrawn from vascular channels such as blood vessels.

What is claimed is:

1. A fluid injection device comprising:
a fluid injection unit having; a fluid chamber body in which a fixed face, a diaphragm facing the fixed face, a fluid chamber having a space surrounded by a side wall continuing the fixed face and the diaphragm, and an inlet channel tube and an outlet channel tube communicating with the fluid chamber are integrally molded; an actuator in close contact with the diaphragm; and a lid body holding the fluid chamber body,
wherein inner surfaces of respective connecting parts of the fixed face, the side wall, and the diaphragm are rounded in molding, and
a volume of the fluid chamber is reduced by the diaphragm, and a pulsating fluid is injected from a nozzle opening provided in an end direction of the outlet channel tube.

2. The fluid injection device according to claim 1, wherein the outlet channel tube is protruded on the fixed face, and the fluid chamber has a rotator shape and the outlet channel tube is provided near a rotational axis of the rotator shape, and
a swirling flow generating part that generates a swirling flow of the fluid around the rotational axis of the rotator shape is provided on the inner surface of the fixed face or the diaphragm.

3. The fluid injection device according to claim 1, wherein the inlet channel tube is provided on the fixed face in parallel to the outlet channel tube.

4. The fluid injection device according to claim 1, wherein the inlet channel tube is protruded on the side wall.

5. The fluid injection device according to claim 4, wherein the outlet channel tube is provided on the fixed face, and the fluid chamber has a rotator shape and the outlet channel tube is provided near a rotational axis of the rotator shape, and
the inlet channel tube is communicated along an inner circumferential surface of the side wall.

6. The fluid injection device according to claim 1, wherein the lid body includes an upper case holding the fixed face and a lower case holding a circumferential edge of the diaphragm, and
projecting portions that respectively cover outer circumferences of side surfaces of the inlet channel tube and the outlet channel tube are provided on the upper case or the lower case.

7. The fluid injection device according to claim 6, wherein the upper case is formed of a metal layer over the fixed face and the side wall, the metal layer being thicker than the diaphragm.

8. The fluid injection device according to claim 7, wherein the metal layer is formed over the fixed face, the side wall, and the circumferential edge of the diaphragm.

9. The fluid injection device according to claim 1, wherein the nozzle opening is formed on an end of the outlet channel tube.

10. The fluid injection device according to claim 9, wherein a reinforcing tube that covers an outer circumferential side surface of the outlet channel tube is further provided.

11. The fluid injection device according to claim 1, wherein the inlet channel tube and the outlet channel tube are provided on the side wall in positions opposed with respect to the fluid chamber.

12. The fluid injection device according to claim 11, wherein the lid body has a nearly circular section shape in a perpendicular direction to a fluid flow direction.

13. The fluid injection device according to claim 1, wherein the actuator is a thin-film piezoelectric element.
